(19) United States
(12) Patent Application Publication
(10) Pub. No.: US 2006/0058831 A1
(43) Pub. Date: Mar. 16, 2006

(54) Inflatable system for cervical dilation and labor induction

(76) Inventor: Jack Atad, Haifa (IL)

Correspondence Address: MARTIN MOYNIHAN;c/o ANTHONY CASTORINA, SUITE 207, 2001 JEFFERSON DAVIS HIGHWAY, ARLINGTON, VA, 22202, US

(21) Appl. No.: 10/938,870

(22) Filed: Sep. 13, 2004

(51) Int. Cl. A61M 29/00

(52) U.S. Cl. 606/193

(57) ABSTRACT

An inflatable system, of between one and three balloons, for cervical dilation and labor induction is provided. The inflatable system may have a uterine balloon, for positioning at a proximal portion of the uterus, with respect to an operator, adjacent to the cervical internal os, the uterine balloon being shaped so as to maximize the pressure against the decidua and the internal cervical os and so as to minimize the pressure on the fetal head. Additionally or alternatively, the inflatable system may have a vaginal balloon, for positioning in the vagina, for applying pressure on the external cervical os. Additionally or alternatively, the inflatable system may have a cervical balloon, for positioning in the cervical canal, the cervical balloon being shaped so as to maximize the contact area with the cervix. The balloons are operative to stimulate the secretion of hormone, by exerting pressure on the proximal decidual surfaces of the uterus and on the cervix, so as to soften and ripen the cervix, cause the cervix to dilate, and induce labor. The balloons, which may have rough external surfaces, in order to keep them anchored in place, may be inflated by the operator, directly after their insertion, or manually and gradually, by the woman herself. Various sensors and other instruments may be used with the inflatable system, to monitor cervical dilation, fetal well-being, and the woman's conditions.

### FIELD AND BACKGROUND OF THE INVENTION

The present invention relates to a system and method of labor induction, and in particular, to a special intracorporeal system, comprising balloons, designed to induce cervical dilation and labor induction, while performing monitoring tasks related to the labor.

There are various reasons to induce labor. For example, when a woman is two or more weeks overdue (postdate), and labor does not start on its own, it may be desirous to induce labor, due to fetal or maternal indications, such as placental dysfunction, pregnancy induced hypertension, Preeclampsia, Diabetes, intra-uterine growth restriction, conditions that may jeopardize fetal well-being, or other conditions that may effect the woman's health.

FIGS. 1A and 1B schematically illustrate woman's reproductive systems under normal conditions and during pregnancy.

FIG. 1A illustrates a woman's reproductive system 10, under normal conditions, showing a vagina 12, a cervix 16, forming a cervical canal 14, an external cervical os 15, an internal cervical os 17, a uterus 18, and an endometrium 21, being the mucous membrane lining the uterus 18.

Additionally, FIG. 1B illustrates a woman's reproductive system 20, during pregnancy, illustrating an amniotic sac 25 containing a fetus 27 having a head 29. A decidua 23 is the mucous membrane lining the uterus 18 in preparation for, and during pregnancy.

Labor may be induced by causing the cervix 16 to soften and open. For example, the pharmaceutical substance, prostaglandin (PG), leads to local biochemical and biophysical alterations in the cervical region that reduce cervical resistance and induce myometrial contractions. Endocervical, or vaginal application of PG, in a gel form, has gained acceptance for priming the cervix 16 before labor induction and for labor induction. A pre-manufactured mixture of 0.5 mg PGE₂ and 2.5 mg triacetin gel, such as Prepidil Gel (supplied by Pharmacia N.V./S.A., For Pharmacia & Upjohn Company, a subsidiary of Pharmacia Corporation, Kalamazoo, Mich. 49001, USA) is now available, and recent trials exhibited no gel-specific problems relating to stability, homogeneity, or sterility.

Currently, the Prepidil Gel is applied by intra-cervical injection using a syringe with a simple canula. However, it is extremely difficult, if not impossible, to administer 3 ml of gel in a strictly endocervical fashion without applying some of the gel retroamniotically, which may cause side effects to uterine hypercontractility, and may lead to fetal distress.

A mechanical device for labor induction is also known. U.S. Pat. No. 4,976,692, to Atad, describes a double-balloon catheter 30, illustrated in FIGS. 2A and 2B, designed to be placed in the woman's reproductive system 20 for about 12 hours. The catheter 30 includes proximal and distal balloons 44 and 42, with respect to an operator, inflatable via lumens 34 and 38. When inserted, the balloon 42 is designed for placement in the uterus 18, for pressing against the internal cervical os 17, and the balloon 44 is designed for placement in the vagina 12, against the external cervical os 15. A section 46 of the catheter 30, having an opening 48, is located between the balloons 44 and 42, in the cervical canal 14.

In accordance with a first embodiment, the catheter 30 is used for inserting a medication, such as Prepidil Gel, via a third lumen 36, in communication with the opening 48. The balloons 44 and 42 apply the gel between the external cervical os 15 and the internal cervical os 17, thus inducing labor. In accordance with a second embodiment, the proximal and distal balloons 44 and 42 alone, without gel, are used to induce labor, by pressing against the openings of the external cervical os 15 and the internal cervical os 17, triggering hormone secretion from the deciduas adjacent to the internal cervical os 17, leading to cervical dilation and labor induction without medication.

However, the double-balloon catheter 30 has certain disadvantages. The distal balloon 42 may press against the fetal head 29 (FIG. 1B). Additionally, the balloons 44 and 42 are not operative in the cervical canal 14, where their triggering action is most needed.

There is thus a need for an inflatable system for cervical dilation and labor induction devoid of these limitations.

### SUMMARY OF THE INVENTION

The present invention successfully addresses the shortcomings of the presently known configurations by providing an inflatable system, of between one and three balloons, for cervical dilation and labor induction. The inflatable system may have a uterine balloon, for positioning at a proximal portion of the uterus, with respect to an operator, adjacent to the cervical internal os, the uterine balloon being shaped so as to maximize the pressure against the decidua and the internal cervical os and so as to minimize the pressure on the fetal head. Additionally or alternatively, the inflatable system may have a vaginal balloon, for positioning in the vagina, for applying pressure on the external cervical os. Additionally or alternatively, the inflatable system may have a cervical balloon, for positioning in the cervical canal, the cervical balloon being shaped so as to maximize the contact area with the cervix. The balloons are operative to stimulate the secretion of hormone, by exerting pressure on the proximal decidual surfaces of the uterus and on the cervix, so as to soften and ripen the cervix, cause the cervix to dilate, and induce labor. The balloons, which may have rough external surfaces, in order to keep them anchored in place, may be inflated by the operator, directly after their insertion, or manually and gradually, by the woman herself. Various sensors and other instruments may be used with the inflatable system, to monitor cervical dilation, fetal well-being, and the woman's conditions.

In accordance with one aspect of the present invention, there is provided an inflatable system for cervical dilation and labor induction, comprising:

a catheter, which defines a coordinate system of x;y axes, a length axis substantially along the x-axis, and proximal and distal ends, with respect to an operator, along the length axis, the catheter comprising:

a uterine balloon having a uterine-balloon height, substantially parallel with the x-axis and a uterine-balloon diameter, substantially parallel with the y-axis, wherein the uterine balloon is designed to inflate so that the uterine-balloon height is no more than about 0.75 of the uterine-balloon diameter; and

a uterine-balloon lumen, in communication with the uterine balloon, via an opening in the uterine balloon lumen,

the catheter being designed for insertion into a reproductive system of a woman, having a vagina, a cervical canal, and a uterus, so that the uterine balloon and the opening in the uterine-balloon lumen are positioned at a proximal portion of the uterus, and the uterine balloon is inflated in the proximal portion of the uterus.

In accordance with an additional aspect of the present invention, the uterine balloon is designed to inflate so that the uterine-balloon height is no more than about 0.50 of the uterine-balloon diameter.

In accordance with an additional aspect of the present invention, the uterine balloon is shaped as a substantially flat disk.

In accordance with an alternative aspect of the present invention, the uterine balloon is shaped as an ellipse.

In accordance with an alternative aspect of the present invention, the uterine balloon is concave, with an apex at a proximal side thereof.

In accordance with an alternative aspect of the present invention, the uterine balloon is shaped as a cone, having a base, at a distal end thereof, parallel with the y-axis, and an apex at a proximal end thereof.

In accordance with an additional aspect of the present invention, the uterine balloon has a surface roughness, for anchoring the uterine balloon in place.

In accordance with an additional aspect of the present invention, the uterine balloon is adapted for inflation by a hand pump, by the woman, after insertion.

In accordance with an additional aspect of the present invention, the catheter further includes a cervical balloon, in communication with a cervical-balloon lumen, via an opening in the cervical-balloon lumen, the catheter being designed for insertion into the reproductive system of the woman, so that the cervical balloon and the opening in the cervical-balloon lumen are positioned within the cervical canal and the cervical balloon is inflated in the cervical canal.

In accordance with an additional aspect of the present invention, the cervical balloon is shaped as a cylinder, having a base parallel with the y-axis.

In accordance with an alternative aspect of the present invention, the cervical balloon extends proximally to the vagina and is shaped as a cone, having a base, at a proximal end thereof, in the vagina, parallel with the y-axis, and an apex at a distal end thereof, in the cervical canal.

In accordance with an additional aspect of the present invention, the cervical balloon has a surface roughness, for anchoring the cervical balloon in place.

In accordance with an additional aspect of the present invention, the cervical balloon is adapted for inflation by a hand pump, by the woman, after insertion.

In accordance with an additional aspect of the present invention, the catheter further includes a vaginal balloon, in communication with a vaginal-balloon lumen, via an opening in the vaginal-balloon lumen, the catheter being designed for insertion into the reproductive system of the woman, so that the vaginal balloon and the opening in the vaginal-balloon lumen are positioned within the vagina, at a distal portion thereof, and the vaginal balloon is inflated in the vagina.

In accordance with an additional aspect of the present invention, the vaginal balloon has a surface roughness, for anchoring the vaginal balloon in place.

In accordance with an additional aspect of the present invention, the vaginal balloon is adapted for inflation by a hand pump, by the woman, after insertion.

In accordance with an additional aspect of the present invention, the inflatable system is designed for insertion by palpation.

In accordance with an additional aspect of the present invention, the inflatable system is designed as a stand-alone system.

In accordance with an additional aspect of the present invention, the inflatable system includes at least one additional lumen, having at least one opening, the at least one additional lumen being operable for inserting at least one device to the reproductive system of the woman.

In accordance with an additional aspect of the present invention, the at least one additional lumen is operable for inserting the at least one device to the uterus.

In accordance with an alternative aspect of the present invention, the at least one additional lumen is operable for inserting the at least one device to the cervical canal.

In accordance with an alternative aspect of the present invention, the at least one additional lumen is operable for inserting the at least one device to the vagina.

In accordance with an additional aspect of the present invention, the at least one device is selected from the group consisting of a device for breaking water, a device for sensing amniotic-fluid temperature, a device for sensing fetal heart-beat, a device for measuring an extent of cervical dilation, a device for measuring a frequency of uterine contractility, a device for measuring an intensity of uterine contractility, a device for amnioscopy, a device for fetoscopy, and a device for scalp blood pH sampling.

In accordance with an additional aspect of the present invention, the at least one device further includes a transmitter for transmitting a measurement extracorporeally.

In accordance with an additional or an alternative aspect of the present invention, the at least one device includes a drug form, designed for passive dispensing of a medication.

In accordance with an additional aspect of the present invention, the passive dispensing of a medication is performed in a manner selected from the group consisting of instantaneous release, delayed release, pulsating release, timed release, and slow release.

In accordance with an alternative aspect of the present invention, the at least one device is designed for electronically-controlled dispensing of a medication.

In accordance with an additional aspect of the present invention, the device for electronically-controlled dispensing of a medication is pre-programmed.

In accordance with an additional aspect of the present invention, the device further includes a receiver and the electronically-controlled dispensing of a medication is performed responsive to an extracorporeal input.

In accordance with an additional aspect of the present invention, the device further includes at least one sensor, and the electronically-controlled dispensing of a medication is performed responsive to a measurement of the sensor, in a closed-loop manner.

In accordance with another aspect of the present invention, there is provided an inflatable system for cervical dilation and labor induction, comprising:

a catheter, which defines a coordinate system of x;y axes, a length axis substantially along the x-axis, and proximal and distal ends, with respect to an operator, along the length axis, the catheter comprising:

a cervical balloon, mounted on the catheter; and

a cervical-balloon lumen, in the catheter, in communication with the cervical-balloon, via an opening in the cervical-balloon lumen,

the catheter being designed for insertion into a reproductive system of a woman, having a vagina, a cervical canal, and a uterus, so that the cervical balloon and the opening in the cervical-balloon lumen are positioned in the cervical canal, and the cervical balloon is inflated in the cervical canal.

In accordance with an additional aspect of the present invention, the cervical balloon is shaped as a cylinder, having a base parallel with the y-axis.

In accordance with an alternative aspect of the present invention, the cervical balloon extends further to the vagina and is shaped as a cone, having a base, at a proximal end thereof, parallel with the y-axis, and an apex at a distal end thereof.

In accordance with an additional aspect of the present invention, the cervical balloon has a surface roughness, for anchoring the cervical balloon in place.

In accordance with an additional aspect of the present invention, the cervical balloon is adapted for inflation by a hand pump, by the woman, after insertion.

In accordance with an additional aspect of the present invention, the catheter further includes a vaginal balloon, in communication with a vaginal-balloon lumen, via an opening in the vaginal-balloon lumen, the catheter being designed for insertion into the reproductive system of the woman, so that the vaginal balloon and the opening in the vaginal-balloon lumen are positioned within the vagina, at a distal portion thereof, and the vaginal balloon is inflated in the vagina.

In accordance with an additional aspect of the present invention, the vaginal balloon has a surface roughness, for anchoring the vaginal balloon in place.

In accordance with an additional aspect of the present invention, the vaginal balloon is adapted for inflation by a hand pump, by the woman, after insertion.

In accordance with an additional aspect of the present invention, the inflatable system is designed for insertion by palpation.

In accordance with an additional aspect of the present invention, the inflatable system is designed as a stand-alone system.

In accordance with an additional aspect of the present invention, the inflatable system includes at least one additional lumen, having at least one opening, the at least one additional lumen being operable for inserting at least one device to the reproductive system of the woman.

In accordance with an additional aspect of the present invention, the at least one additional lumen is operable for inserting the at least one device to the uterus.

In accordance with an alternative aspect of the present invention, the at least one additional lumen is operable for inserting the at least one device to the cervical canal.

In accordance with an alternative aspect of the present invention, the at least one additional lumen is operable for inserting the at least one device to the vagina.

In accordance with an additional aspect of the present invention, the at least one device is selected from the group consisting of a device for breaking water, a device for sensing amniotic-fluid temperature, a device for sensing fetal heart-beat, a device for measuring an extent of cervical dilation, a device for measuring a frequency of uterine contractility, a device for measuring an intensity of uterine contractility, a device for amnioscopy, a device for fetoscopy, and a device for scalp blood pH sampling.

In accordance with an additional aspect of the present invention, the at least one device further includes a transmitter for transmitting a measurement extracorporeally.

In accordance with an additional or an alternative aspect of the present invention, the at least one device includes a drug form, designed for passive dispensing of a medication.

In accordance with an additional aspect of the present invention, the passive dispensing of a medication is performed in a manner selected from the group consisting of instantaneous release, delayed release, pulsating release, timed release, and slow release.

In accordance with an alternative aspect of the present invention, the at least one device is designed for electronically-controlled dispensing of a medication.

In accordance with an additional aspect of the present invention, the device for electronically-controlled dispensing of a medication is pre-programmed.

In accordance with an additional aspect of the present invention, the at least one device further includes a receiver and the electronically-controlled dispensing of a medication is performed responsive to an extracorporeal input.

In accordance with an additional aspect of the present invention, the at least one device further includes at least one sensor, and the electronically-controlled dispensing of a medication is performed responsive to a measurement of the sensor, in a closed-loop manner.

In accordance with still another aspect of the present invention, there is provided a method for cervical dilation and labor induction, comprising:

providing an inflatable system for cervical dilation and labor induction, which comprises:
a catheter, which defines a coordinate system of x;y axes, a length axis substantially along the x-axis, and proximal and distal ends, with respect to an operator, along the length axis, the catheter comprising:
a uterine balloon having a uterine-balloon height, substantially parallel with the x-axis and a uterine-balloon diameter, substantially parallel with the y-axis, wherein the uterine balloon is designed to inflate so that the uterine-balloon height is no more than about 0.75 of the uterine-balloon diameter; and
a uterine-balloon lumen, in communication with the uterine balloon, via an opening in the uterine balloon lumen;

inserting the catheter into a reproductive system of a woman, having a vagina, a cervical canal, and a uterus, so that the uterine balloon and the opening in the uterine-balloon lumen are positioned at a proximal portion of the uterus; and

inflating the uterine balloon in the proximal portion of the uterus.

In accordance with yet another aspect of the present invention, there is provided a method for cervical dilation and labor induction, comprising:

providing an inflatable system for cervical dilation and labor induction, which comprises:
a catheter, which defines a coordinate system of x;y axes, a length axis substantially along the x-axis, and proximal and distal ends, with respect to an operator, along the length axis, the catheter comprising:
a cervical balloon, mounted on the catheter; and
a cervical-balloon lumen, in the catheter, in communication with the cervical-balloon, via an opening in the cervical-balloon lumen;

inserting the catheter into a reproductive system of a woman, having a vagina, a cervical canal, and a uterus, so that the cervical balloon and the opening in the cervical-balloon lumen are positioned in the cervical canal; and

inflating the cervical balloon in the cervical canal.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1A-1B are schematic diagrams of a woman's reproduction system;

FIGS. 2A-2B schematically illustrate an inflatable system for cervical dilation and labor induction, as known;

FIGS. 3A-3O schematically illustrate inflatable systems for cervical dilation and labor induction, in accordance with the present invention;

FIGS. 4A-4M schematically illustrate various balloon arrangements of an inflatable system for cervical dilation and labor induction, in accordance with the present invention;

FIGS. 5A-5J schematically illustrate different devices used in conjunction with an inflatable system for cervical dilation and labor induction, in accordance with the present invention;

FIGS. 6A-6D schematically illustrate different designs for electronic devices of the present invention; and

FIGS. 7A-7B schematically illustrate a single cervical balloon for cervical dilation and labor induction, having a rough surface, in accordance with the present invention.

### DESCRIPTION OF THE PREFERRED EMBODIMENTS

The present invention relates to an inflatable system, of between one and three balloons, for cervical dilation and labor induction. The inflatable system may have a uterine balloon, for positioning at a proximal portion of the uterus, with respect to an operator, adjacent to the cervical internal os, the uterine balloon being shaped so as to maximize the pressure against the decidua and the internal cervical os and so as to minimize the pressure on the fetal head. Additionally or alternatively, the inflatable system may have a vaginal balloon, for positioning in the vagina, for applying pressure on the external cervical os. Additionally or alternatively, the inflatable system may have a cervical balloon, for positioning in the cervical canal, the cervical balloon being shaped so as to maximize the contact area with the cervix. The balloons are operative to stimulate the secretion of hormone, by exerting pressure on the proximal decidual surfaces of the uterus and on the cervix, so as to soften and ripen the cervix, cause the cervix to dilate, and induce labor. The balloons, which may have rough external surfaces, in order to keep them anchored in place, may be inflated by the operator, directly after their insertion, or manually and gradually, by the woman herself. Various sensors and other instruments may be used with the inflatable system, to monitor cervical dilation, fetal well-being, and the woman's conditions.

The principles and operation of the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways.

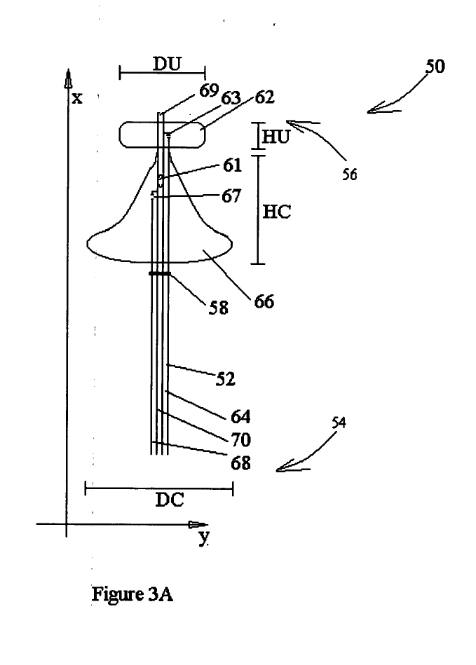

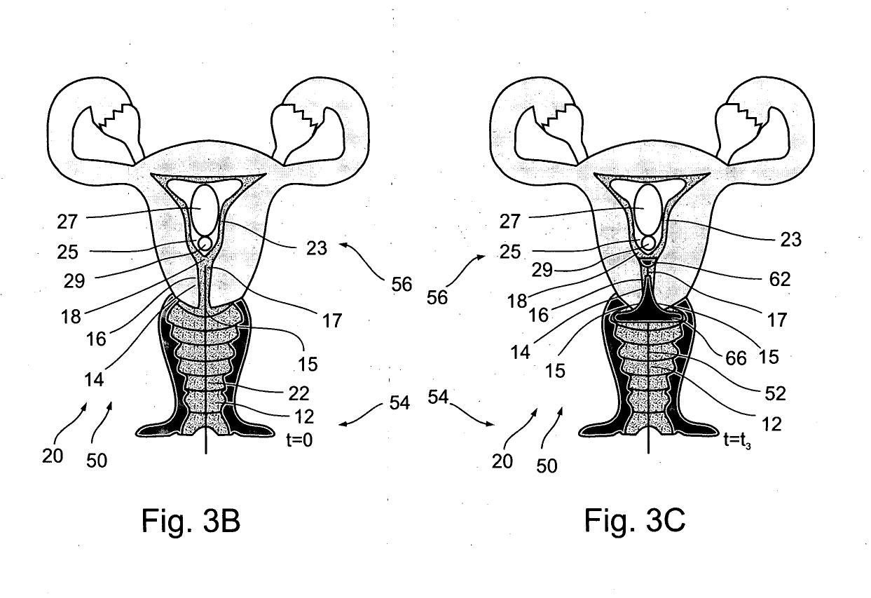

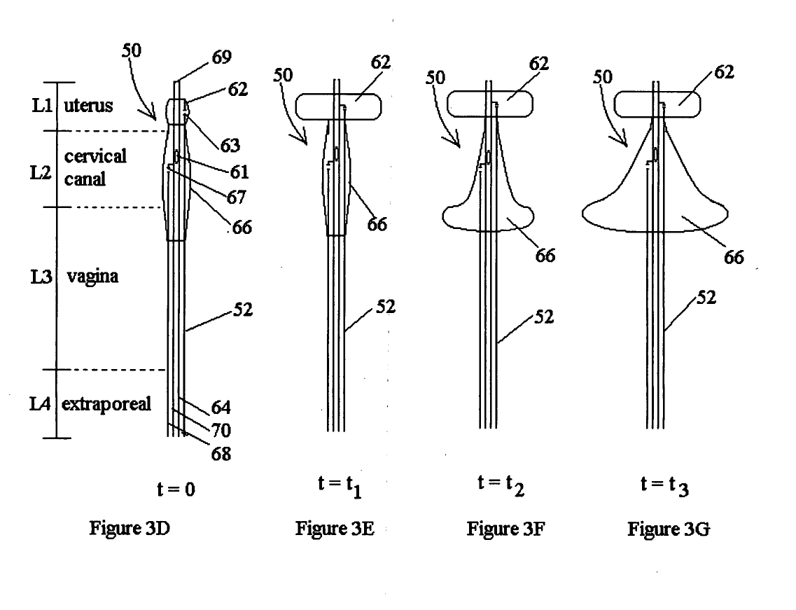

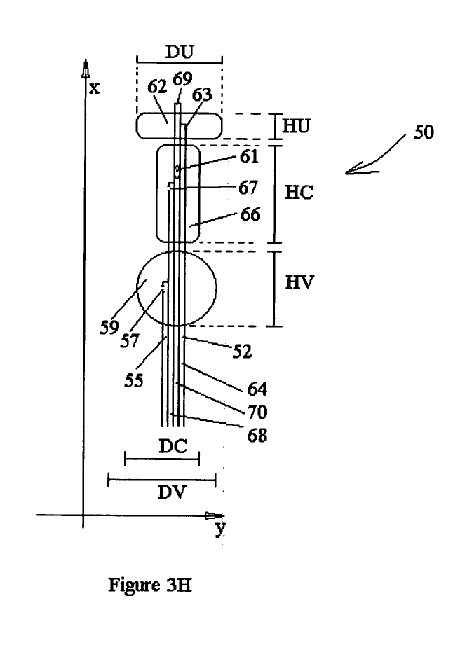

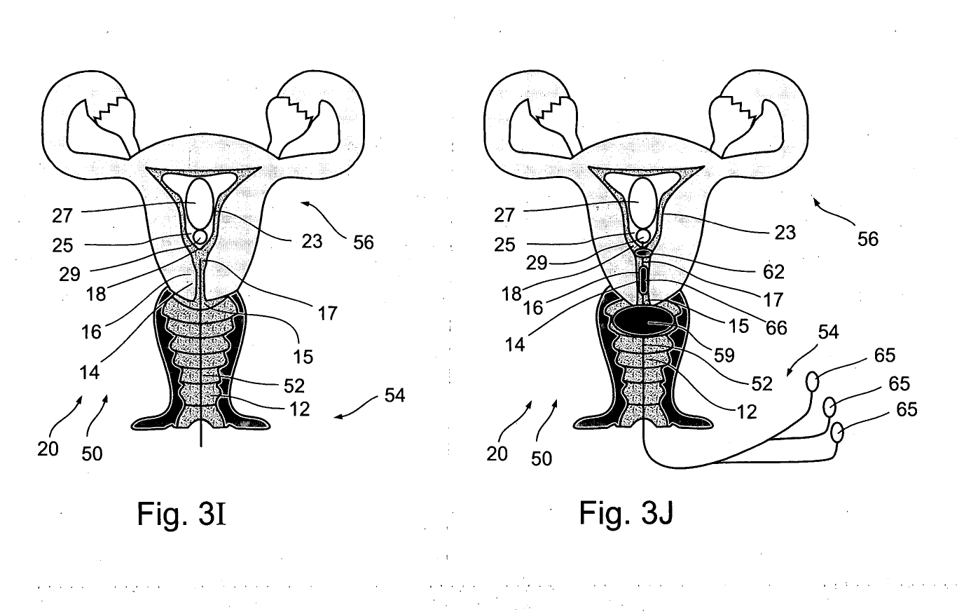

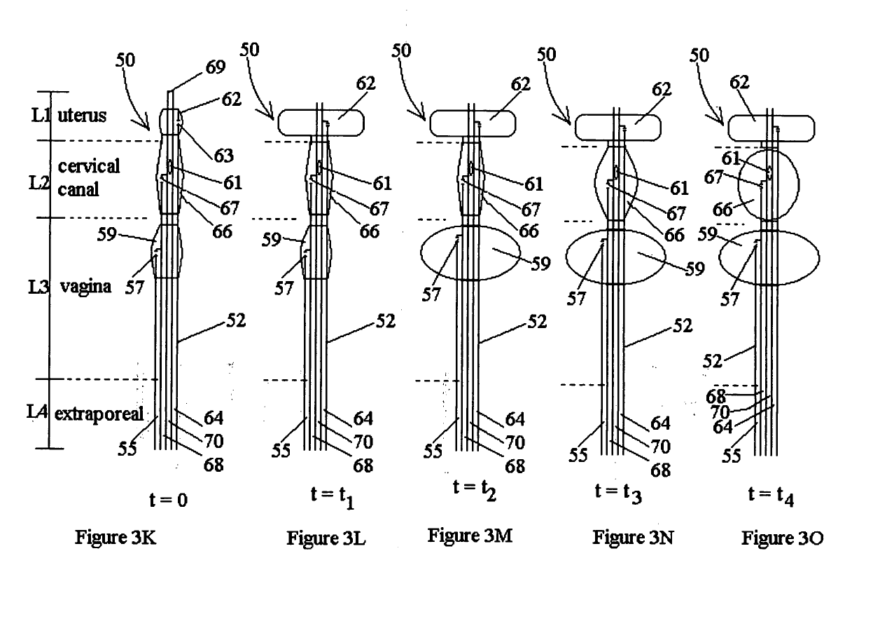

Referring now to the drawings, FIGS. 3A-3O schematically illustrate inflatable systems 50 for cervical dilation and labor induction, in accordance with the present invention.

As seen in FIG. 3A, the inflatable system 50 for cervical dilation and labor induction includes a catheter 52, which defines a coordinate system of x;y axes, and has a length axis substantially along the x-axis, and proximal and distal ends, 54 and 56, respectively, with respect to an operator (not seen), along the length axis. The catheter 52 includes:

a uterine balloon 62 having a uterine-balloon height HU, substantially parallel with the x-axis, and a uterine-balloon diameter DU, substantially parallel with the y-axis, the uterine balloon 62 being shaped so that the uterine-balloon height HU is no more than about 0.75, and preferably, no more than 0.50 of the uterine-balloon diameter DU; and

a uterine-balloon lumen 64, in fluid communication with the uterine balloon 62, via an opening 63, in the uterine-balloon lumen 64.

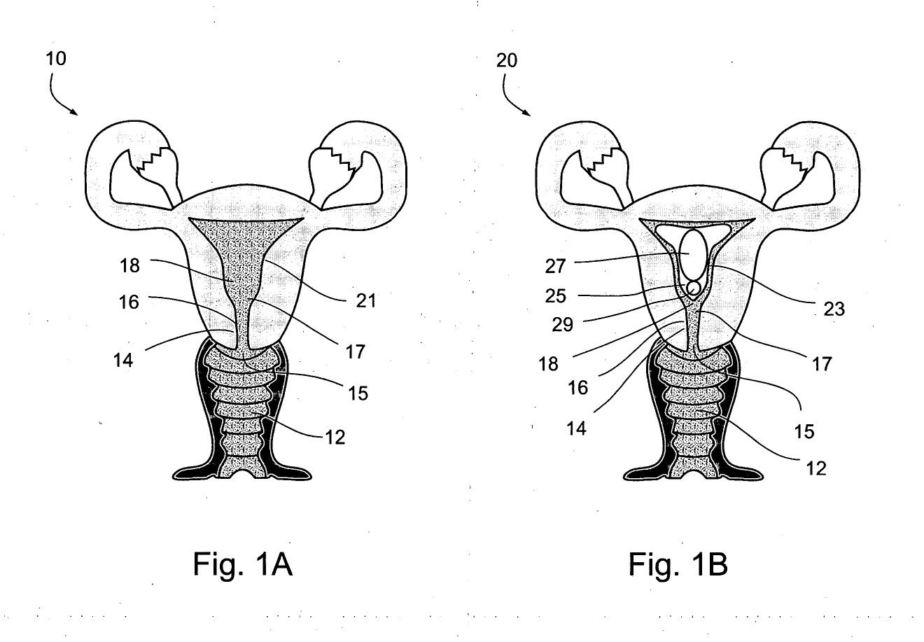

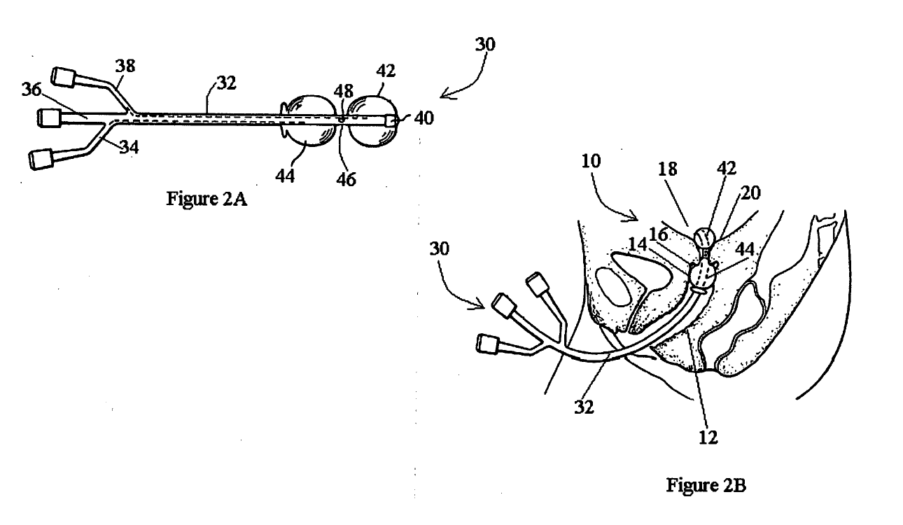

In accordance with a first embodiment of the present invention, the catheter 52 is designed for insertion into a woman's reproductive system 20 (FIG. 1B), so that the uterine balloon 62 and the opening 63 are positioned at a proximal portion of the uterus 18. The specific dimensional relationship of the uterine balloon 62 is designed to maximize the pressure against the decidua 23 (FIG. 1B) and the internal cervical os 17, and to minimize the pressure on the fetal head 29.

Additionally, in accordance with a preferred embodiment of the present invention, the catheter 52 includes a cervical balloon 66, in communication with a cervical-balloon lumen 68, via an opening 67, in the cervical-balloon lumen 68, the cervical balloon 66 having a cervical-balloon height HC, substantially parallel with the x-axis, and a cervical-balloon diameter DC, substantially parallel with the y-axis. The catheter 52 is designed for insertion into the woman's reproductive system 20, so that the cervical balloon 66 and the opening 67 are positioned within the cervical canal 14, and the cervical balloon 66 is inflated in the cervical canal 14. In accordance with an embodiment of the present invention, the cervical balloon 66 may further extend to a distal portion of the vagina 12. The cervical balloon 66 is designed for applying pressure on the cervix 16. It will be appreciated that the pressure on the cervix 16 may further include pressure on the internal cervical os 17 and on the external cervical os 15.

FIGS. 3B illustrates the inflatable system 50, inserted within the woman's reproductive system 20, prior to inflation.

FIG. 3C illustrates the inflatable system 50, when fully inflated, with the inflated uterine balloon 62 in the proximal portion of the uterus 18, and the inflated cervical balloon 66 in the cervical canal 14, extending to the distal portion of the vagina 12. As seen, the uterine balloon 62 applies pressure on the decidua 23 and the internal cervical ox 17 but not on the fetal head 29. The cervical balloon 66 applies pressure on the cervix 16, the internal cervical os 17, and preferably also the external cervical os 15.

Thus, as seen from FIG. 3C, the uterine balloon 62 is operative to separate the amniotic sac 25 from the decidua 23, thereby stimulating endogenous hormone secretion of PG from the decidua. The hormone secretion by the decidua 23 is operative to soften and ripen the cervical canal 14 and induce labor.

Similarly, the cervical balloon 66 is operative to stimulate hormone secretion by the cervix 16. Again, the hormone secretion is operative to soften and ripen the cervical canal 14 and induce labor.

For maximum effect, the following criteria are met:

i. the uterine balloon 62 is shaped so as to apply maximum pressure on the decidua 23 and on the internal cervical os 17;

ii. the uterine balloon 62 is further shaped so as to minimize the pressure on the fetus, inflating sideways, in the ±y direction, with minimal inflation distally, towards the fetal head, in the +x direction, so that the uterine-balloon height HU is no more than about 0.75, and preferably, no more than 0.50 of the uterine-balloon diameter DU; and

iii. the cervical balloon 66 is shaped so a to have a maximum contact area with the cervix 16, for applying pressure on the cervix 16, preferably, including the internal cervical os 17 and the external cervical os 15, to accelerate cervical dilation.

Preferably, the inflatable system 50 is inflated in stages, as illustrated in FIGS. 3C-3F, as follows:

FIG. 3D illustrates the inflatable system 50 at a time t=0, prior to inflation;

FIG. 3E illustrates the inflatable system 50 at a first time step, time t=1, when only the uterine balloon 62 is inflated;

FIG. 3F illustrates the inflatable system 50 at a second time step, time t=2, when the uterine balloon 62 is fully inflated, and the cervical balloon 66 begins to inflate; and

FIG. 3G illustrates the inflatable system 50 at a third time step, time =3, when both the uterine balloon 62 and the cervical balloon 66 are fully inflated.

For withdrawal, the inflatable system 50 is deflated, returning to the state of t=0 (FIGS. 3B and 3D).

As seen in FIG. 3A, the catheter 52 may include a palpable demarcation ring 58 located on the proximal side of the cervical balloon 66. The ring 58 enables correct positioning of the catheter 52 by palpation, even without cervical visualization. Thus, the catheter 52 may be inserted into the woman's reproductive system 20 (FIGS. 1B and 3B-3C) while the woman lies supine in bed, thereby avoiding the discomfort with the lithotomy position and the insertion of a vaginal speculum.

It will be appreciated that the system 50 may be designed as a stand-alone system, so the woman may move freely with the system 50 inserted.

In accordance with a preferred embodiment of the present invention, seen in FIG. 3H, the inflatable system 50 for cervical dilation and labor induction may include three balloons, as follows:

The uterine balloon 62, shaped so that the uterine-balloon height HU is no more than about 0.75, and preferably, no more than 0.50 of the uterine-balloon diameter DU, the uterine balloon 62 being in fluid communication with the uterine balloon lumen 64, via the opening 63, and the uterine balloon 62 being adapted for positioning at the proximal portion of the uterus 18, for applying pressure on the internal cervical os 17 and the decidua 23. For example, the uterine-balloon diameter DU may be 6 cm and the uterine-balloon height HU may be 2 cm. It will be appreciated that other dimensions are also possible.

Additionally, the inflatable system 50 may include the cervical balloon 66, in fluid communication with the cervical-balloon lumen 68, via the opening 67, the cervical balloon 66 being adapted for positioning within the cervical canal 14, for applying pressure on the cervix 16. It will be appreciated that the pressure on the cervix 16 may further include pressure on the internal cervical os 17 and on the external cervical os 15. Preferably the cervical balloon 66 is shaped so that the cervical-balloon diameter DC is greater than the cervical-balloon height HC. For example, the cervical-balloon diameter may be 6 cm, and the cervical-balloon height HC may be 4 cm. It will be appreciated that other dimensions are also possible. Alternatively, a round cervical balloon may be used.

Furthermore, the inflatable system 50 may include a vaginal balloon 59, in fluid communication with a vaginal-balloon lumen 55, via an opening 57, the vaginal balloon 59 being adapted for positioning within the vagina 12, at a distal portion thereof, for pressing against the external cervical os 15. Preferably, a vaginal-balloon diameter DV is greater than a vaginal-balloon height HV. For example, the vaginal-balloon diameter DV may be 6 cm, and the vaginal-balloon height HV may be 3 cm. It will be appreciated that other dimensions are also possible. Alternatively, a round balloon may be used.

FIG. 3I schematically illustrates the three-balloon inflatable system 50, inserted within the woman's reproductive system 20, prior to inflation.

FIG. 3J schematically illustrates the three-balloon inflatable system 50, inserted within the woman's reproductive system 20, when fully inflated.

In accordance with the preferred embodiment of the present invention, of the three-balloon inflatable system 50, illustrated in FIG. 3J, the uterine balloon 62 and the vaginal balloon 59 are operative to anchor the cervical balloon 66 in place and to enhance hormonal secretion by applying pressure on the decidual 23, the internal cervical os 17, and the external cervical os 15. At the same time, the cervical balloon 66, anchored within the cervical canal 14, may accelerate cervical dilation, by applying pressure on the cervix 16, and preferably also, the internal cervical os 17 and on the external cervical os 15.

FIGS. 3K-3O schematically illustrate the stages of inflation of the three-balloon system 50 of FIG. 3H, in accordance with a preferred embodiment of the present invention.

As seen in FIG. 3K, for insertion, the inflatable system 50 is fully deflated.

As seen in FIG. 3L, at the first time step, time t=1, only the uterine balloon 62 is inflated;

As seen in FIG. 3M, at the second time step, time t=2, both the uterine balloon 62 and the vaginal balloon 59 are inflated.

As seen in FIG. 3N, at the third time step, time t=3, both the uterine balloon 62 and the vaginal balloon 59 are fully inflated, and the cervical balloon 66 begins to inflate.

As seen in FIG. 3O, at the fourth time step, time t=4, both the uterine balloon 62 and the vaginal balloon 59 are fully inflated, and the cervical balloon 66 is inflated further.

For withdrawal, the inflatable system 50 is deflated, returning to the state of t=0 (FIGS. 3I and 3K).

In accordance with the present invention, the inflation of the balloons of the inflatable system 50 may be performed by the doctor or midwife (not shown) directly after their insertion. Alternatively, the inflation of the balloons of the inflatable system 50 may be performed by the woman, via hand pumps 65 (FIG. 3J), in a gradual manner, for example over several hours. Alternatively still, uterine balloon 62 and vagina balloon 59 may be inflated by the doctor or midwife, while the inflation of the cervical balloon 66 may be performed by the woman, for example, via one of hand pumps 65 (FIG. 3J).

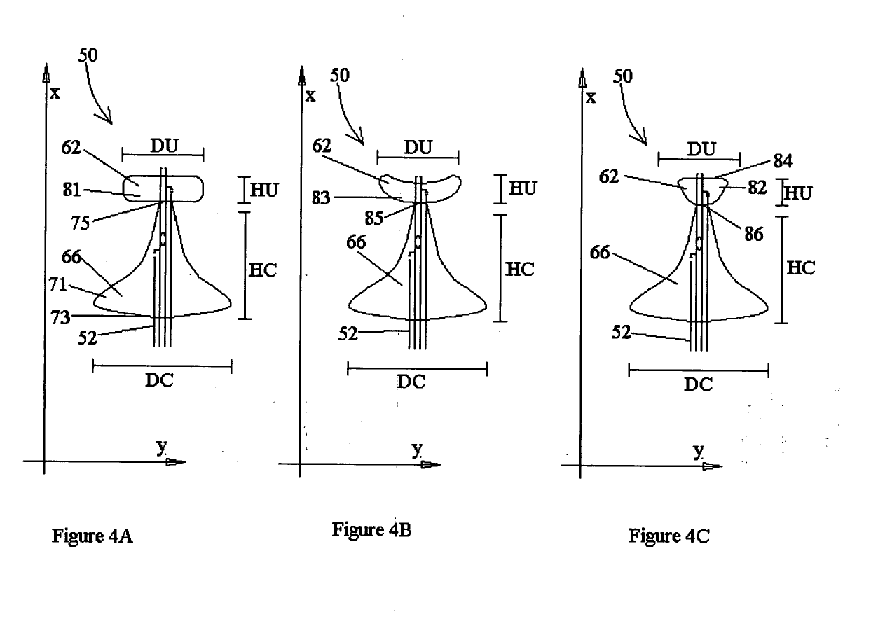

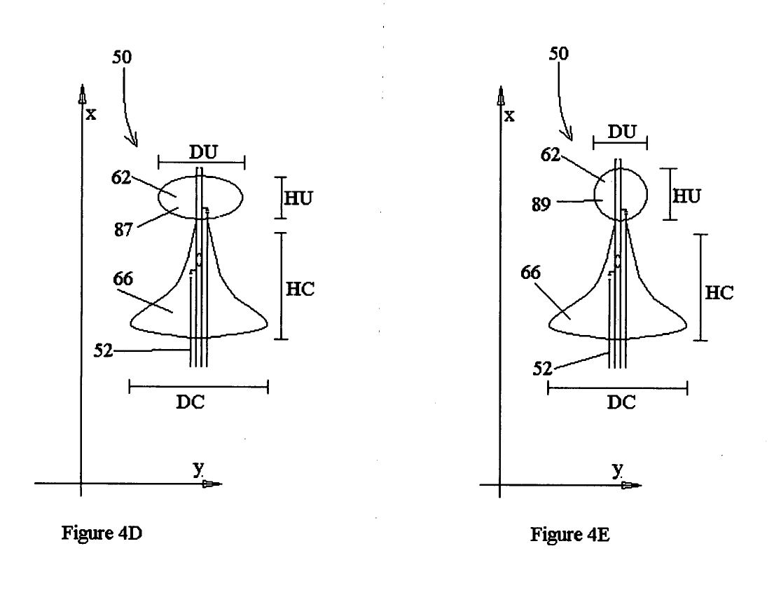

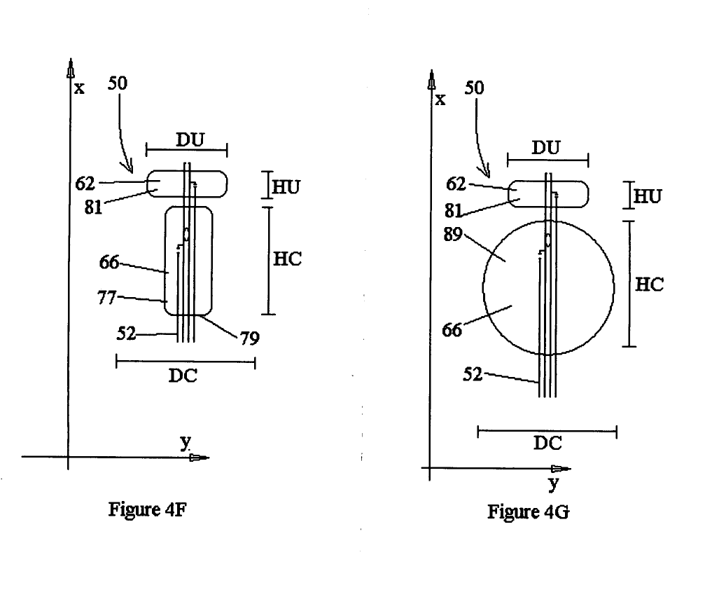

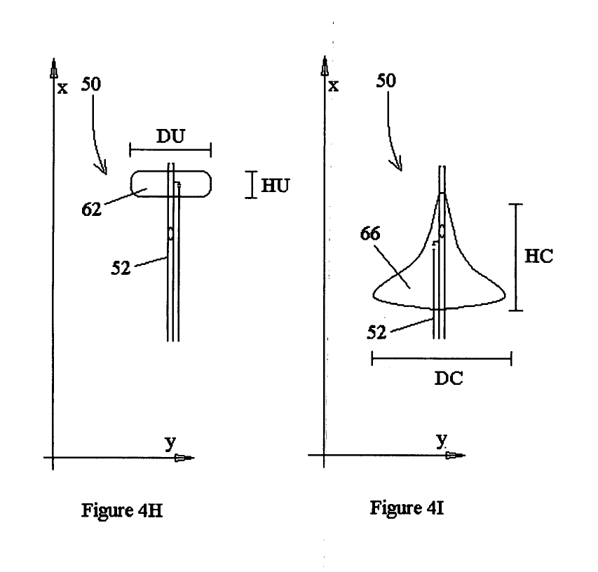

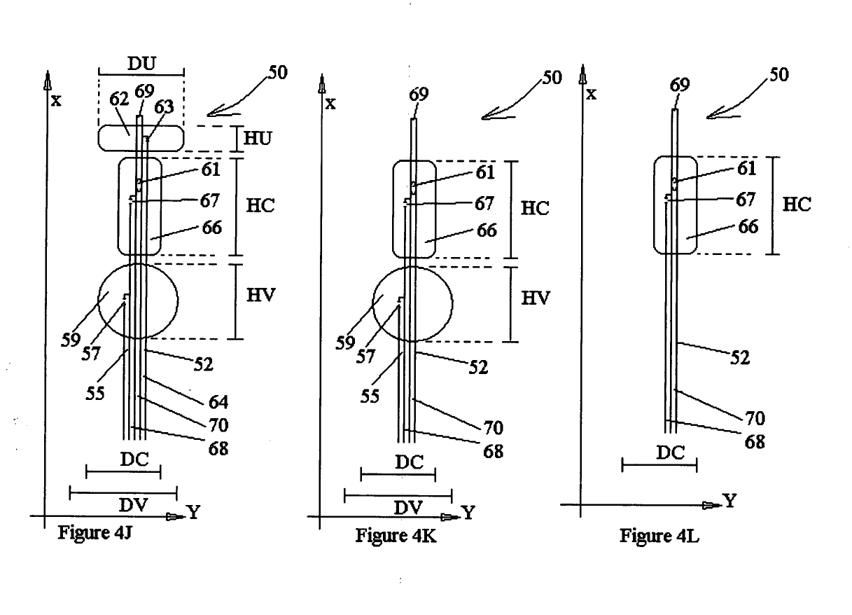

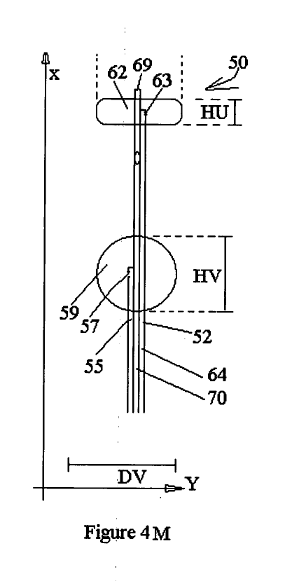

Referring further to the drawings, FIGS. 4A-4M schematically illustrate various balloon arrangements of the inflatable system 50 for cervical dilation and labor induction, designed to meet criteria (i)-(iii) hereinabove, in accordance with the present invention.

With regard to the uterine balloon 62, shaped so that the uterine-balloon height HU is no more than about 0.75, and preferably, no more than 0.50 of the uterine-balloon diameter DU, in order to maximize the pressure against the decidua 23 and the internal cervical os 17, and to minimize the pressure on the fetal head 29:
i. as seen in FIG. 4A, the uterine balloon 62 may be shaped as a flat disk 81;
ii. as seen in FIG. 4B, the uterine balloon 62 may be shaped as a dome 83, with an apex 85, at a proximal side thereof;
iii. as seen in FIG. 4C, the uterine balloon 62 may be shaped as a cone 82, having a base 84, at a distal end thereof, parallel with the y-axis, and an apex 86, at a proximal end thereof; and
iv. as seen in FIG. 4D, the uterine balloon 62 may be shaped as an ellipse 87.

However, in accordance with an alternative embodiment, seen in FIG. 4E, the uterine balloon 62 may be shaped as a ball 89, expanding equally in the x and y directions, for example, as taught in U.S. Pat. No. 4,976,892, to Atad, whose disclosure is incorporated herein by reference.

With regard to the cervical balloon 66, designed to apply pressure on the cervix 16, the internal cervical os 17, and preferably also the external cervical os 15:
i. as seen in FIG. 4F, the cervical balloon 66 may be shaped as a cylinder 77, having a base 79, parallel with the y-axis; and
ii. as seen in FIGS. 4A and 3C, the cervical balloon 66 may extend proximally to the vagina 12 and may be shaped as a cone 71, having a wide base 73, parallel with the y-axis, in the vaginal portion of the balloon, where resistance to the inflation is relatively low, and a narrow portion in the cervical portion of the balloon 66, where resistance to the inflation is relatively high, tapering to an apex 75, at a distal end thereof.

However, in accordance with an alternative embodiment, seen in FIG. 4G, the cervical balloon 66 may be shaped as a ball 89, expanding equally in the x and y directions.

It will be appreciated that in accordance with alternative embodiments of the present invention, seen in FIGS. 4H and 4I, only the uterine balloon 62, or only the cervical balloon 66 may be used.

FIG. 4J schematically illustrates the three balloon arrangement of FIG. 3H, wherein the uterine balloon 62 is shaped as a disk, the cervical balloon 66 is shaped as a cylinder, and the vaginal balloon 59 is shaped as a round ball.

It will be appreciated that even for the three-balloon system, the uterine balloon 62 may be shaped as any one of the uterine balloons of FIGS. 4A-4E, and the cervical balloon 66 and the vaginal balloon 59 may be shaped as disks, cylinders, round balls, or other shapes.

FIG. 4K schematically illustrates a two balloon arrangement of only the vaginal balloon 59 and the cervical balloon 66. It will be appreciated that the cervical balloon 66 and the vaginal balloon 59 may be shaped as disks, cylinders, round balls, or other shapes.

FIG. 4L schematically illustrates a single balloon arrangement of only the cervical balloon 66. It will be appreciated that the cervical balloon 66 may be shaped as a disk, a cylinder, a round ball, or another shape

FIG. 4M schematically illustrates a two balloon arrangement of only the uterine balloon 62, shaped in accordance with the present invention, as taught by any one of FIGS. 4A-4E, and the vaginal balloon 59, shaped as a disk, a cylinder, a round ball, or another shape

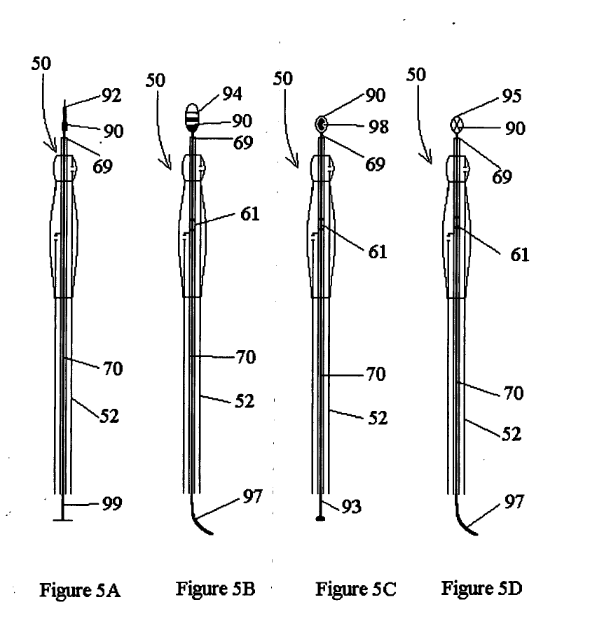

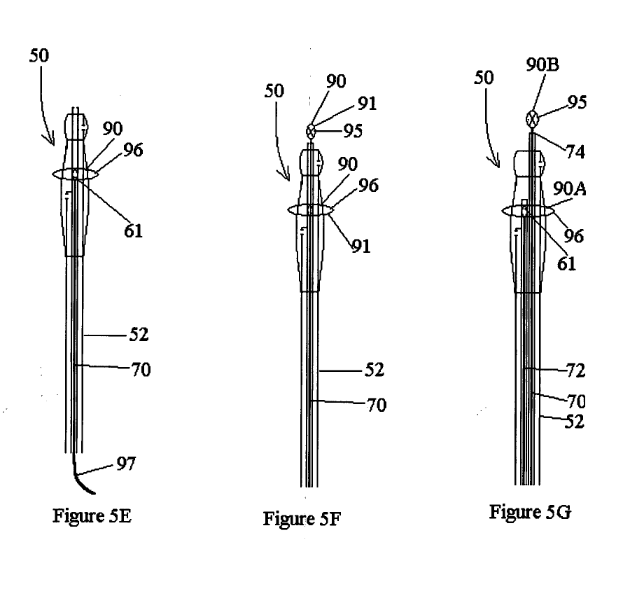

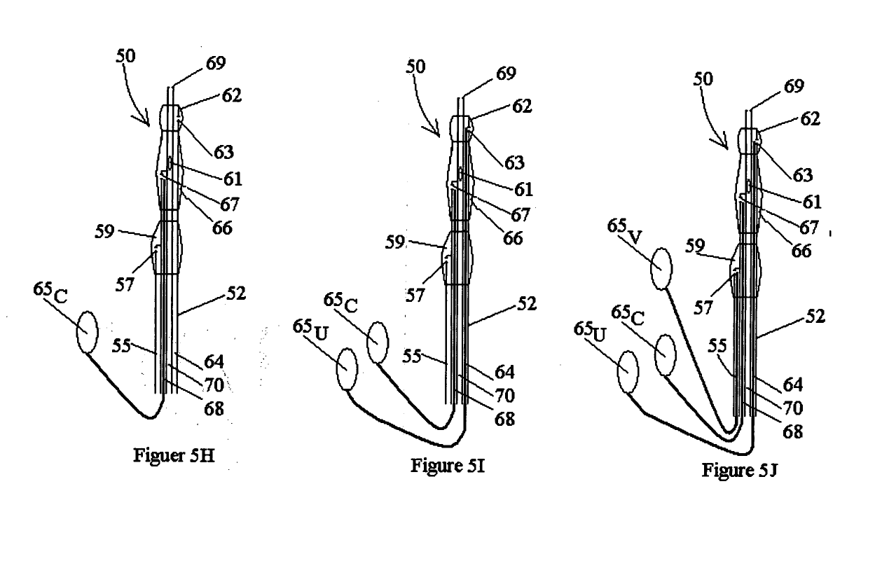

Referring further to the drawings, FIGS. 5A-5J schematically illustrate different devices, when used in conjunction with the inflatable system 50 for cervical dilation and labor induction, in accordance with the present invention.

Preferably, the system 50 includes at least one additional lumen 70, having at least one opening 69, to the uterus 18, and possibly an additional opening 61, to the cervical canal 14. The at least one additional lumen 70 is operable for inserting at least one device 90 to the woman's reproductive system 20.

In accordance with an aspect of the present invention, the at least one additional lumen 70 is operable for inserting the at least one device 90 to the uterus 18, via the opening 69.

Alternatively, the at least one additional lumen 70 is operable for inserting the at least one device 90 to the cervical canal 14, via the opening 61.

As seen in FIG. 5A, the at least one device 90 may be a device 92 for breaking water, operable via the opening 69 to the uterus 18. Device 92 may be operated as a plunger-like device, using a handle 99. After the puncture, the device 92 is withdrawn.

As seen in FIG. 5B, the at least one device 90 may be a sensor 94, for example, a sensor for sensing amniotic-fluid temperature, or for sensing fetal heart-beat, preferably operable via the opening 69 to the uterus 18. The measurements may be transmitted extracorporeally in a wireless manner, via RF or IR. Alternatively, a wire 97 may be used.

It will be appreciated that the device 90 may be selected from the group consisting of device for breaking water, a device for sensing amniotic-fluid temperature, a device for sensing fetal heart-beat, a device for measuring an extent of cervical dilation, a device for measuring a frequency of uterine contractility, a device for measuring an intensity of uterine contractility, a device for amnioscopy, a device for fetoscopy, a device for scalp blood pH sampling, and other measuring devices as known.

As seen in FIG. 5C, the at least one device 90 may include a drug form 98, designed for passive dispensing of a medication. The drug form may be inserted with a syringe 93, and the syringe 93 may then be withdrawn.

The passive dispensing of a medication may be by instantaneous release, delayed release, pulsating release, timed release, slow release, or another release form, as known, operable via the opening 69 to the uterus 18, or via the openings 61 to the cervical canal 14.

As seen in FIG. 5D, the at least one device 90 may be an electronically-controlled device 95, for dispensing of a medication, operable via the opening 69 to the uterus 18, or via the openings 61 to the cervical canal 14. The device 95 may be pre-programmed, or may receive instructions in a wireless manner from an extracorporeal station (not shown), via RF or IR. Alternatively, a wire 97 may be used.

As seen in FIG. 5E, the at least one device 90 may include a device 96 for measuring an extent of cervical dilation, operable via the opening 61 to the cervical canal 14. The measurements may be transmitted extracorporeally in a wireless manner, via RF or IR. Alternatively, a wire 97 may be used.

As seen in FIG. 5F, the at least one device 90 may include a device 91 comprising both device 96 for measuring an extent of cervical dilation, and device 95 for electronically-controlled dispensing of a medication, for a closed loop operation, wherein the medication is dispensed responsive to the measurements of the extent of cervical dilation.

As seen in FIG. 5G, the inflatable system 50 may include still an additional lumen 72, having at least one opening 74, to the uterus 18 or to the cervical canal 14. Thus, two devices 90A and 90B may be used simultaneously. For example, device 90A may be the device 96 for measuring an extent of cervical dilation, inserted in the lumen 70 and operable via the opening 61 to the cervical canal 14, while device 90B may be the electronically-controlled device 95, for dispensing of a medication, operable via the opening 74. In accordance with the present invention, the electronically-controlled device 95 for dispensing of a medication may operate responsive to the measurements of the device 96 for measuring an extent of cervical dilation. The communication may be from the device 96 to an extracorporeal station (not shown), and from the extracorporeal station to the device 95, in a wireless manner, or via a wire. Alternatively, the communication between the two devices may be direct, in a closed-loop manner.

FIG. 5H schematically illustrates the three-balloon arrangement of FIG. 3H, with a single hand pump 65_{C}, for example, in communication with the cervical balloon 66. The single hand pump 65_{C} is designed for use by the woman, for manually and gradually inflating the cervical balloon 66, over a period of several hours, in order to hasten cervical dilation.

FIG. 5I schematically illustrates the three-balloon arrangement of FIG. 3H, with two hand pumps 65_{C} and 65_{U}, for example, in communication with the cervical balloon 66 and the uterine balloon 62. Again, the hand pumps are designed for use by the woman, for manually and gradually inflating the uterine balloon 62 then the cervical balloon 66, in order to hasten cervical dilation. Preferably, the uterine balloon is inflated over a period of several minutes and the cervical balloon is inflated over a period of several hours.

FIG. 5J schematically illustrates the three balloon arrangement of FIG. 3H, with the three hand pumps 65_{C}, 65_{U}, and 65_{U}, in communication with the cervical balloon 66, the uterine balloon 62, and the vaginal balloon 59. The hand pumps are designed for use by the woman, for manually and gradually inflating first the uterine balloon 62 then the vaginal balloon 59, and last, the cervical balloon 66, in order to hasten cervical dilation. Preferably, the uterus and vaginal balloons are inflated over a period of several minutes and the cervical balloon is inflated over a period of several hours.

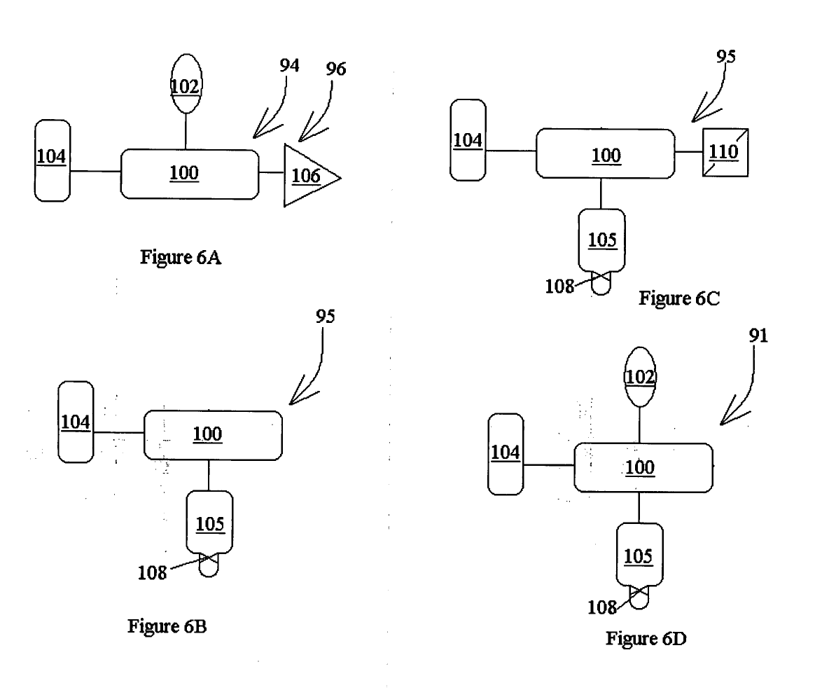

Referring further to the drawings, FIGS. 6A-6D schematically illustrate different designs for the devices 90, when electronic devices, in accordance with the present invention.

FIG. 6A illustrates the device 94 (FIG. 5B) for sensing amniotic-fluid temperature, or fetal heart-beat, and (or) the device 96 (FIGS. 5E, 5G), for measuring the extent of cervical dilation.

Accordingly, the devices 94 and (or) 96 include:

a sensor 102, for performing the sensing or the measuring;

preferably also, a transmitter 106, such as an RF or an IR transmitter, for transmitting the sensing or the measuring, extracorporeally;

a power source 104, for powering the device 90; and

a dedicated circuitry 100, as needed.

FIG. 6B illustrates the electronically-controlled device 95 (FIGS. 5D, 5G), for dispensing of a medication.

Accordingly, the device 95 includes a drug pouch 105, controlled by an electronically controlled valve 108, which is powered by the power source 104. The dedicated circuitry 100, may be included, as needed, and may be preprogrammed for a specific drug-release schedule.

FIG. 6C illustrates the electronically-controlled device 95 (FIGS. 5D, 5G), for dispensing of a medication, designed for extracorporeal communication.

Accordingly, the device 95 includes the drug pouch 105, controlled by the electronically controlled valve 108, which is powered by the power source 104, and which dispenses the drug, responsive to extracorporeal instructions, as received via a receiver 110. The dedicated circuitry 100, may be included, as needed.

FIG. 6D illustrates the electronically-controlled device 91 (FIG. 5F), for dispensing of a medication, designed for a closed-loop operation, responsive to a measurement of the sensor 102. Alternatively the closed loop operation may be used with the two devices of FIG. 5G.

Accordingly, the device 95 includes the drug pouch 105, controlled by the electronically-controlled valve 108, which is powered by the power source 104, and which dispenses the drug, responsive to input of the sensor 102. The dedicated circuitry 100, may be included, as needed. The sensor 102 may be, for example, a sensor for amniotic-fluid temperature, a sensor for fetal heart-beat, or a device for measuring the extent of cervical dilation.

It will be appreciated that in any one of FIGS. 6A-6D, the dedicated circuitry 100 may be a controller or a microcomputer.

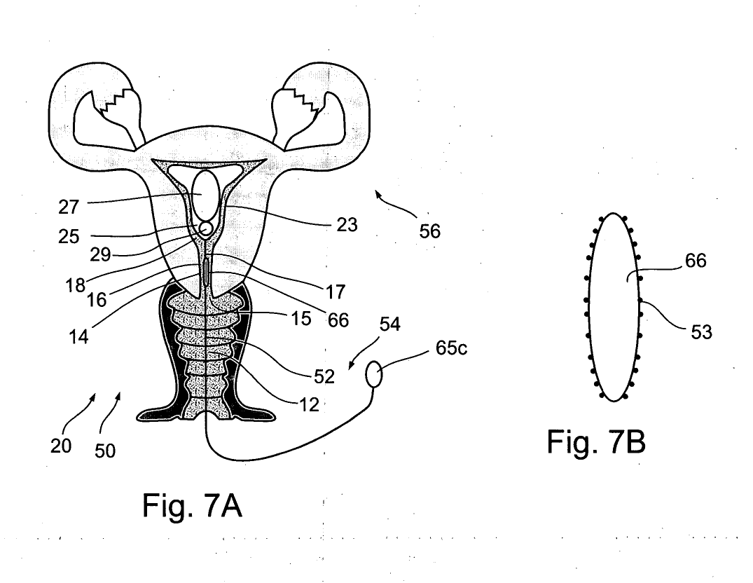

Referring further to the drawings, FIGS. 7A and 7B schematically illustrate the single cervical balloon 66 of FIG. 4L and 5H, with a surface roughness, in accordance with the present invention.

At times, it may be desirous for the inflatable system 50 to include only the cervical balloon 66. This may happen, for example, that spontaneous labor has started, but the woman wishes to speed it up. Using the hand-pump 65_{C}, she may inflate the cervical balloon 66 gradually, so as to continuously increase the pressure on the cervix 16 and hasten its dilation. However, without being anchored by the vaginal balloon 59, or held in place by the uterine balloon 62, the cervical balloon 59 may slide out of position. Therefore, as seen in FIG. 7B, when used alone, the cervical balloon 66 may be provided with a surface roughness 73 that helps maintain it in place.

It will be appreciated that the surface roughness 73 may be similarly applied to the uterine balloon 62 and (or) to the vaginal balloon 59. Additionally, the surface roughness 73 may be applied whether the inflatable system 50 is of a single balloon, two balloons, or three balloons.

As used herein the term “about” refers to ±20%.

It is expected that during the life of this patent, many relevant inflatable systems for cervical dilation may be developed and the scope of the term inflatable system for cervical dilation and labor induction is intended to include all such new technologies a priori.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, a citation or identification of any reference in this application shall not be construed as an admission that such reference is available as prior art to the present invention.

What is claimed is:

1. An inflatable system for cervical dilation and labor induction, comprising:
a catheter, which defines a coordinate system of x;y axes, a length axis substantially along said x-axis, and proximal and distal ends, with respect to an operator, along said length axis, said catheter comprising:
a uterine balloon having a uterine-balloon height, substantially parallel with said x-axis and a uterine-balloon diameter, substantially parallel with said y-axis, wherein said uterine balloon is designed to inflate so that said uterine-balloon height is no more than about 0.75 of said uterine-balloon diameter; and
a uterine-balloon lumen, in communication with said uterine balloon, via an opening in said uterine balloon lumen,
said catheter being designed for insertion into a reproductive system of a woman, having a vagina, a cervical canal, and a uterus, so that said uterine balloon and said opening in said uterine-balloon lumen are positioned at a proximal portion of said uterus, and said uterine balloon is inflated in said proximal portion of said uterus.

2. The inflatable system of claim 1, wherein said uterine balloon is designed to inflate so that said uterine-balloon height is no more than about 0.50 of said uterine-balloon diameter.

3. The inflatable system of claim 1, wherein said uterine balloon is shaped as a substantially flat disk.

4. The inflatable system of claim 1, wherein said uterine balloon is shaped as an ellipse.

5. The inflatable system of claim 1, wherein said uterine balloon is concave, with an apex at a proximal side thereof.

6. The inflatable system of claim 1, wherein said uterine balloon is shaped as a cone, having a base, at a distal end thereof, parallel with said y-axis, and an apex at a proximal end thereof.

7. The inflatable system of claim 1, wherein said uterine balloon has a surface roughness, for anchoring said uterine balloon in place.

8. The inflatable system of claim 1, wherein said uterine balloon is adapted for inflation by a hand pump, by the woman, after insertion.

9. The inflatable system of claim 1, wherein said catheter further includes a cervical balloon, in communication with a cervical-balloon lumen, via an opening in said cervical-balloon lumen, said catheter being designed for insertion into the reproductive system of the woman, so that said cervical balloon and said opening in said cervical-balloon lumen are positioned within said cervical canal and said cervical balloon is inflated in said cervical canal.

10. The inflatable system of claim 9, wherein said cervical balloon is shaped as a cylinder, having a base parallel with said y-axis.

11. The inflatable system of claim 9, wherein said cervical balloon extends proximally to said vagina and is shaped as a cone, having a base, at a proximal end thereof, in said vagina, parallel with said y-axis, and an apex at a distal end thereof, in said cervical canal.

12. The inflatable system of claim 9, wherein said cervical balloon has a surface roughness, for anchoring said cervical balloon in place.

13. The inflatable system of claim 9, wherein said cervical balloon is adapted for inflation by a hand pump, by the woman, after insertion.

14. The inflatable system of claim 1, wherein said catheter further includes a vaginal balloon, in communication with a vaginal-balloon lumen, via an opening in said vaginal-balloon lumen, said catheter being designed for insertion into the reproductive system of the woman, so that said vaginal balloon and said opening in said vaginal-balloon lumen are positioned within said vagina, at a distal portion thereof, and said vaginal balloon is inflated in said vagina.

15. The inflatable system of claim 14, wherein said vaginal balloon has a surface roughness, for anchoring said vaginal balloon in place.

16. The inflatable system of claim 14, wherein said vaginal balloon is adapted for inflation by a hand pump, by the woman, after insertion.

17. The inflatable system of claim 1, designed for insertion by palpation.

18. The inflatable system of claim 1, designed as a stand-alone system.

19. The inflatable system of claim 1, and further including at least one additional lumen, having at least one opening, said at least one additional lumen being operable for inserting at least one device to the reproductive system of the woman.

20. The inflatable system of claim 19, wherein said at least one additional lumen is operable for inserting said at least one device to said uterus.

21. The inflatable system of claim 19, wherein said at least one additional lumen is operable for inserting said at least one device to said cervical canal.

22. The inflatable system of claim 19, wherein said at least one additional lumen is operable for inserting said at least one device to said vagina.

23. The inflatable system of claim 19, wherein said at least one device is selected from the group consisting of a device for breaking water, a device for sensing amniotic-fluid temperature, a device for sensing fetal heart-beat, a device for measuring an extent of cervical dilation, a device for measuring a frequency of uterine contractility, a device for measuring an intensity of uterine contractility, a device for amnioscopy, a device for fetoscopy, and a device for scalp blood pH sampling.

24. The inflatable system of claim 19, wherein said at least one device further includes a transmitter for transmitting a measurement extracorporeally.

25. The inflatable system of claim 19, wherein said at least one device includes a drug form, designed for passive dispensing of a medication.

26. The inflatable system of claim 25, wherein said passive dispensing of a medication is performed in a manner selected from the group consisting of instantaneous release, delayed release, pulsating release, timed release, and slow release.

27. The inflatable system of claim 19, wherein said at least one device is designed for electronically-controlled dispensing of a medication.

28. The inflatable system of claim 27, wherein said device for electronically-controlled dispensing of a medication is pre-programmed.

29. The inflatable system of claim 27, wherein said device further includes a receiver and said electronically-controlled dispensing of a medication is performed responsive to an extracorporeal input.

30. The inflatable system of claim 27, wherein said device further includes at least one sensor, and said electronically-controlled dispensing of a medication is performed responsive to a measurement of said sensor, in a closed-loop manner.

31. An inflatable system for cervical dilation and labor induction, comprising:
a catheter, which defines a coordinate system of x;y axes, a length axis substantially along said x-axis, and proximal and distal ends, with respect to an operator, along said length axis, said catheter comprising:
a cervical balloon, mounted on said catheter; and
a cervical-balloon lumen, in said catheter, in communication with said cervical-balloon, via an opening in said cervical-balloon lumen,
said catheter being designed for insertion into a reproductive system of a woman, having a vagina, a cervical canal, and a uterus, so that said cervical balloon and said opening in said cervical-balloon lumen are positioned in said cervical canal, and said cervical balloon is inflated in said cervical canal.

32. The inflatable system of claim 31, wherein said cervical balloon is shaped as a cylinder, having a base parallel with said y-axis.

33. The inflatable system of claim 31, wherein said cervical balloon extends further to said vagina and is shaped as a cone, having a base, at a proximal end thereof, parallel with said y-axis, and an apex at a distal end thereof.

34. The inflatable system of claim 31, wherein said cervical balloon has a surface roughness, for anchoring said cervical balloon in place.

35. The inflatable system of claim 31, wherein said cervical balloon is adapted for inflation by a hand pump, by the woman, after insertion.

36. The inflatable system of claim 31, wherein said catheter further includes a vaginal balloon, in communication with a vaginal-balloon lumen, via an opening in said vaginal-balloon lumen, said catheter being designed for insertion into the reproductive system of the woman, so that said vaginal balloon and said opening in said vaginal-balloon lumen are positioned within said vagina, at a distal portion thereof, and said vaginal balloon is inflated in said vagina.

37. The inflatable system of claim 36, wherein said vaginal balloon has a surface roughness, for anchoring said vaginal balloon in place.

38. The inflatable system of claim 36, wherein said vaginal balloon is adapted for inflation by a hand pump, by the woman, after insertion.

39. The inflatable system of claim 31, designed for insertion by palpation.

40. The inflatable system of claim 31, designed as a stand-alone system.

41. The inflatable system of claim 31, and further including at least one additional lumen, having at least one opening, said at least one additional lumen being operable for inserting at least one device to the reproductive system of the woman.

42. The inflatable system of claim 41, wherein said at least one additional lumen is operable for inserting said at least one device to said uterus.

43. The inflatable system of claim 41, wherein said at least one additional lumen is operable for inserting said at least one device to said cervical canal.

44. The inflatable system of claim 41, wherein said at least one additional lumen is operable for inserting said at least one device to said vagina.

45. The inflatable system of claim 41, wherein said at least one device is selected from the group consisting of a device for breaking water, a device for sensing amniotic-fluid temperature, a device for sensing fetal heart-beat, a device for measuring an extent of cervical dilation, a device for measuring a frequency of uterine contractility, a device for measuring an intensity of uterine contractility, a device for amnioscopy, a device for fetoscopy, and a device for scalp blood pH sampling.

46. The inflatable system of claim 41, wherein said at least one device further includes a transmitter for transmitting a measurement extracorporeally.

47. The inflatable system of claim 41, wherein said at least one device includes a drug form, designed for passive dispensing of a medication.

48. The inflatable system of claim 47, wherein said passive dispensing of a medication is performed in a manner selected from the group consisting of instantaneous release, delayed release, pulsating release, timed release, and slow release.

49. The inflatable system of claim 41, wherein said at least one device is designed for electronically-controlled dispensing of a medication.

50. The inflatable system of claim 49, wherein said device for electronically-controlled dispensing of a medication is pre-programmed.

51. The inflatable system of claim 49, wherein said at least one device further includes a receiver and said electronically-controlled dispensing of a medication is performed responsive to an extracorporeal input.

52. The inflatable system of claim 49, wherein said at least one device further includes at least one sensor, and said electronically-controlled dispensing of a medication is performed responsive to a measurement of said sensor, in a closed-loop manner.

53. A method for cervical dilation and labor induction, comprising:
providing an inflatable system for cervical dilation and labor induction, which comprises:
a catheter, which defines a coordinate system of x;y axes, a length axis substantially along said x-axis, and proximal and distal ends, with respect to an operator, along said length axis, said catheter comprising:
a uterine balloon having a uterine-balloon height, substantially parallel with said x-axis and a uterine-balloon diameter, substantially parallel with said y-axis, wherein said uterine balloon is designed to inflate so that said uterine-balloon height is no more than about 0.75 of said uterine-balloon diameter; and
a uterine-balloon lumen, in communication with said uterine balloon, via an opening in said uterine balloon lumen;
inserting said catheter into a reproductive system of a woman, having a vagina, a cervical canal, and a uterus, so that said uterine balloon and said opening in said uterine-balloon lumen are positioned at a proximal portion of said uterus; and
inflating said uterine balloon in said proximal portion of said uterus.

54. The method of claim 53, wherein said uterine balloon is designed to inflate so that said uterine-balloon height is no more than about 0.50 of said uterine-balloon diameter.

55. The method of claim 53, wherein said uterine balloon is shaped as a substantially flat disk.

56. The method of claim 53, wherein said uterine balloon is shaped as an ellipse.

57. The method of claim 53, wherein said uterine balloon is concave, with an apex at a proximal side thereof.

58. The method of claim 53, wherein said uterine balloon is shaped as a cone, having a base, at a distal end thereof, parallel with said y-axis, and an apex at a proximal end thereof.

59. The method of claim 53, wherein said uterine balloon has a surface roughness, for anchoring said uterine balloon in place.

60. The method of claim 53, wherein said uterine balloon is adapted for inflation by a hand pump, by the woman, after insertion.

61. The method of claim 53, wherein said catheter further includes a cervical balloon, in communication with a cervical-balloon lumen, via an opening in said cervical-balloon lumen, said catheter being designed for insertion into the reproductive system of the woman, so that said cervical balloon and said opening in said cervical-balloon lumen are positioned within said cervical canal and said cervical balloon is inflated in said cervical canal.

62. The method of claim 61, wherein said cervical balloon is shaped as a cylinder, having a base parallel with said y-axis.

63. The method of claim 61, wherein said cervical balloon extends proximally to said vagina and is shaped as a cone, having a base, at a proximal end thereof, in said vagina, parallel with said y-axis, and an apex at a distal end thereof, in said cervical canal.

64. The method of claim 61, wherein said cervical balloon has a surface roughness, for anchoring said cervical balloon in place.

65. The method of claim 61, wherein said cervical balloon is adapted for inflation by a hand pump, by the woman, after insertion.

66. The method of claim 53, wherein said catheter further includes a vaginal balloon, in communication with a vaginal-balloon lumen, via an opening in said vaginal-balloon lumen, said catheter being designed for insertion into the reproductive system of the woman, so that said vaginal balloon and said opening in said vaginal-balloon lumen are positioned within said vagina, at a distal portion thereof, and said vaginal balloon is inflated in said vagina.

67. The method of claim 66, wherein said vaginal balloon has a surface roughness, for anchoring said vaginal balloon in place.

68. The method of claim 66, wherein said vaginal balloon is adapted for inflation by a hand pump, by the woman, after insertion.

69. The method of claim 53, designed for insertion by palpation.

70. The method of claim 53, designed as a stand-alone system.

71. The method of claim 53, and further including at least one additional lumen, having at least one opening, said at least one additional lumen being operable for inserting at least one device to the reproductive system of the woman.

72. The method of claim 71, wherein said at least one additional lumen is operable for inserting said at least one device to said uterus.

73. The method of claim 71, wherein said at least one additional lumen is operable for inserting said at least one device to said cervical canal.

74. The method of claim 71, wherein said at least one additional lumen is operable for inserting said at least one device to said vagina.

75. The method of claim 71, wherein said at least one device is selected from the group consisting of a device for breaking water, a device for sensing amniotic-fluid temperature, a device for sensing fetal heart-beat, a device for measuring an extent of cervical dilation, a device for measuring a frequency of uterine contractility, a device for measuring an intensity of uterine contractility, a device for amnioscopy, a device for fetoscopy, and a device for scalp blood pH sampling.

76. The method of claim 71, wherein said at least one device further includes a transmitter for transmitting a measurement extracorporeally.

77. The method of claim 71, wherein said at least one device includes a drug form, designed for passive dispensing of a medication.

78. The method of claim 77, wherein said passive dispensing of a medication is performed in a manner selected from the group consisting of instantaneous release, delayed release, pulsating release, timed release, and slow release.

79. The method of claim 71, wherein said at least one device is designed for electronically-controlled dispensing of a medication.

80. The method of claim 79, wherein said device for electronically-controlled dispensing of a medication is pre-programmed.

81. The method of claim 79, wherein said device further includes a receiver and said electronically-controlled dispensing of a medication is performed responsive to an extracorporeal input.

82. The method of claim 79, wherein said device further includes at least one sensor, and said electronically-controlled dispensing of a medication is performed responsive to a measurement of said sensor, in a closed-loop manner.

83. A method for cervical dilation and labor induction, comprising:
providing an inflatable system for cervical dilation and labor induction, which comprises:
a catheter, which defines a coordinate system of x;y axes, a length axis substantially along said x-axis, and proximal and distal ends, with respect to an operator, along said length axis, said catheter comprising:
a cervical balloon, mounted on said catheter; and
a cervical-balloon lumen, in said catheter, in communication with said cervical-balloon, via an opening in said cervical-balloon lumen;
inserting said catheter into a reproductive system of a woman, having a vagina, a cervical canal, and a uterus, so that said cervical balloon and said opening in said cervical-balloon lumen are positioned in said cervical canal; and
inflating said cervical balloon in said cervical canal.

84. The method of claim 83, wherein said cervical balloon is shaped as a cylinder having a base parallel with said y-axis.

85. The method of claim 83, wherein said cervical balloon extends further to said vagina and is shaped as a cone, having a base, at a proximal end thereof, parallel with said y-axis, and an apex at a distal end thereof.

86. The method of claim 83, wherein said cervical balloon has a surface roughness, for anchoring said cervical balloon in place.

87. The method of claim 83, wherein said cervical balloon is adapted for inflation by a hand pump, by the woman, after insertion.

88. The method of claim 83, wherein said catheter further includes a vaginal balloon, in communication with a vaginal-balloon lumen, via an opening in said vaginal-balloon lumen, said catheter being designed for insertion into the reproductive system of the woman, so that said vaginal balloon and said opening in said vaginal-balloon lumen are positioned within said vagina, at a distal portion thereof, and said vaginal balloon is inflated in said vagina.

89. The method of claim 88, wherein said vaginal balloon has a surface roughness, for anchoring said vaginal balloon in place.

90. The method of claim 88, wherein said vaginal balloon is adapted for inflation by a hand pump, by the woman, after insertion.

91. The method of claim 83, designed for insertion by palpation.

92. The method of claim 83, designed as a stand-alone system.

93. The method of claim 83, and further including at least one additional lumen, having at least one opening, said at least one additional lumen being operable for inserting at least one device to the reproductive system of the woman.

94. The method of claim 93, wherein said at least one additional lumen is operable for inserting said at least one device to said uterus.

95. The method of claim 93, wherein said at least one additional lumen is operable for inserting said at least one device to said cervical canal.

96. The method of claim 93, wherein said at least one additional lumen is operable for inserting said at least one device to said vagina.

97. The method of claim 93, wherein said at least one device is selected from the group consisting of a device for breaking water, a device for sensing amniotic-fluid temperature, a device for sensing fetal heart-beat, a device for measuring an extent of cervical dilation, a device for measuring a frequency of uterine contractility, a device for measuring an intensity of uterine contractility, a device for amnioscopy, a device for fetoscopy, and a device for scalp blood pH sampling.

98. The method of claim 93, wherein said at least one device further includes a transmitter for transmitting a measurement extracorporeally.

99. The method of claim 93, wherein said at least one device includes a drug form, designed for passive dispensing of a medication.

100. The method of claim 99, wherein said passive dispensing of a medication is performed in a manner selected from the group consisting of instantaneous release, delayed release, pulsating release, timed release, and slow release.

101. The method of claim 93, wherein said at least one device is designed for electronically-controlled dispensing of a medication.

102. The method of claim 101, wherein said device for electronically-controlled dispensing of a medication is pre-programmed.

103. The method of claim 101, wherein said at least one device further includes a receiver and said electronically-controlled dispensing of a medication is performed responsive to an extracorporeal input.

104. The method of claim 101, wherein said at least one device further includes at least one sensor, and said electronically-controlled dispensing of a medication is performed responsive to a measurement of said sensor, in a closed-loop manner.
